(19) United States
(12) Patent Application Publication
(10) Pub. No.: US 2011/0008821 A1
(43) Pub. Date: Jan. 13, 2011

(54) LABELED MOLECULE FOR PROGNOSING TUMOR GRADE OF HEAD AND NECK CANCER AND METHOD FOR THE SAME

(76) Inventors: Ann-Joy CHENG, Linkou Shiang (TW); Joseph T. Chang, Linkou Shiang (TW); Ching-Chi Chiu, Daliao Shiang (TW)

Correspondence Address: Muncy, Geissler, Olds & Lowe, PLLC, 4000 Legato Road, Suite 310, FAIRFAX, VA, 22033, US

(21) Appl. No.: 12/889,114

(22) Filed: Sep. 23, 2010

Related U.S. Application Data
(62) Division of application No. 12/117,001, filed on May 8, 2008.

(30) Foreign Application Priority Data
Jan. 11, 2008 (TW) 97101258

(51) Int. Cl. C12Q 1/02

(52) U.S. Cl. 435/29

(57) ABSTRACT

The present invention discloses a labeled molecule for prognosing the tumor grade of head and neck cancer and a method for the same, wherein a 78-kDA glucose regulated protein (GRP78) is adopted as a labeled molecule of head and neck cancer. GRP78 is much more overexpressed in head and neck cancer cells than in non-cancer cells. GRP78 also has relation with tumor size, tumor depth and tumor metastasis. Therefore, the present invention uses GRP78 overexpression as a reference for tumor grading of head and neck cancer.

### CROSS REFERENCE TO RELATED APPLICATIONS

This application is a Divisional of co-pending application Ser. No. 12/117,001, filed on 8 May 2008, and for which priority is claimed under 35 U.S.C. §120; and this application claims priority of Application No. 97101258 filed in Taiwan on 11 Jan. 2008 under 35 U.S.C. §119; the entire contents of all of which are hereby incorporated by reference.

### BACKGROUND OF THE INVENTION

1. Field of the Invention

The present invention relates to an application of a 78-kDA glucose regulated protein (GRP78), particularly to a GRP78-containing labeled molecule for prognosing the tumor grade of head and neck cancer and a method for the same.

2. Description of the Related Art

The 78-kDA glucose regulated protein (GRP78), also known as hsp70-5, hspA5 or Bip, is one member of the heat shock protein 70 (HSP70) family. GRP78 is a functional protein, which implements folding up a newly-synthesized protein to have an appropriate conformation in the endoplasmic reticulum. According to previous researches, a crisis of cells, such as a hypoxia state or an ultraviolet radiation, will trigger GRP78 to assist in the degradation of the incorrectly folded protein. Therefore, GRP78 is thought to be a stress sensor of the endoplasmic reticulum, which functions as the cyto-protection and anti-apoptosis mechanisms of cells.

It is also found in some researches that GRP78 can function as the labeled protein of breast cancer. GRP78 can help cells survive in a glucose-deficiency environment. Some researches show: a patient having overexpressed GRP78 has a higher recurrence rate no matter in what stage of cancer. Therefore, it is thought that GRP78 helps breast cancer cells resist against chemotherapy. This finding gives physicians a very important therapeutic reference, and unnecessary medicine and useless therapy can thus be avoided if GRP78 concentration is tested before chemotherapy.

At present, none labeled molecule can be clinically used to determine the tumor grade of head and neck cancer, nor is molecular medicine for an overexpressed gene designed to cure head and neck cancer. In fact, the current technology is still indefinite about whether GRP78 can be used in tumor grading.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIG. 1 is a diagram of electrophoregrams showing that GRP78 is overexpressed in cancer tissues.

### SUMMARY OF THE INVENTION

The primary objective of the present invention is to provide a labeled molecule for prognosing the tumor grade of head and neck cancer and a method for the same, wherein a 78-kDA glucose regulated protein (GRP78) is used to estimate the tumor grade of head and neck cancer, whereby physicians can adopt proper measures to promote the therapeutic effect of head and neck cancer.

The present invention is based on the facts that GRP78 expression in head and neck cancer cells is much higher than the expression in non-cancer cells and that GRP78 expression correlates with clinical malignant indications, such as tumor size, tumor depth, lymph metastasis, etc., wherein the above mentioned facts are obtained via analyzing the relative GRP78 expression in head and neck cancer tissue and normal tissue. Thus, the present invention adopts GRP78 as a labeled molecule for prognosing the tumor grade of head and neck cancer.

Below, detailed description, in cooperation with the drawing, is used to further demonstrate the objectives, characteristics and efficacies of the present invention.

### DETAILED DESCRIPTION OF THE INVENTION

The present invention adopts GRP78 as a labeled molecule for head and neck cancer, which is clinically related with head and neck cancer and overexpressed in head and neck cancer. Below, clinical experiments will be analyzed to verify the theory and efficacies of the present invention.

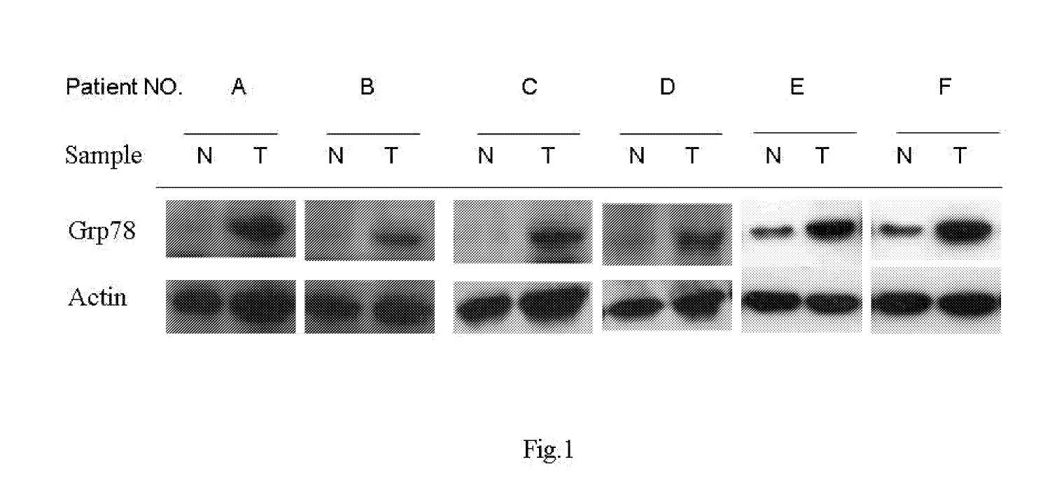

In the present invention, cancer tissues of head and neck cancer patients (Patients A-F) are compared with normal tissues. A protein analysis is used to detect GRP78 expressions in head and neck cancer tissues and normal tissues, and a western blot analysis method is used to perform the protein analysis. Refer to FIG. 1. GRP78 is overexpressed on the electrophoregrams of the head and neck cancer specimens, wherein N denotes a normal tissue, and T denotes a cancer tissue. A cytoskeleton protein (Actin) is used in the control groups. It is found in FIG. 1: GRP78 is overexpressed in the cancer tissue of a patient no matter in protein or RNA (ribonucleic acid).

Table.1 shows the characteristics of the research subjects. The subjects include: 54 males (91.5%) and 5 females (8.5%). The ages range from 29 to 77 years old with a mean of 49. Among them, 37 persons are habituated to alcohol, and 51 persons are habituated to cigarette, and 53 persons are habituated to areca. About 80% of patients have the habit of alcohol drinking, cigarette smoking or areca chewing. Among 56 pairs of specimens, GRP78 is overexpressed in the cancer tissues of 34 patients; the ratio of GRP 78 overexpression is as high as 61%.

TABLE 1

| | | |
|---|---|---|
| | Count of persons | |
| | (59 persons | |
| Characteristics | totally) | Percentage |

| | | | |
|---|---|---|---|
| Sex | Male | 54 | 91.5% |
| | Female | 5 | 8.5% |
| Age | <40 years old | 12 | 20.3% |
| | 40-50 years old | 21 | 35.6% |
| | 51-60 years old | 15 | 25.5% |
| | 61-70 years old | 8 | 13.5% |
| | >70 years old | 3 | 5.1% |
| Addiction | Alcohol | 37 | 62.7% |
| | Cigarette | 51 | 86.4% |
| | Areca | 53 | 89.8% |

Table.2 shows the clinical statistics about GRP78 overexpression and pathological characteristics. It is found in Table.2: GRP78 overexpression has statistical correlations with T stage (P=0.035), N stage (P=0.01), overall stage (P=0.034), tumor depth (P=0.035) and extracapsular spread in lymph nodes (P=0.036). The correlations between GRP78 overexpression and tumor area, tumor stage and tumor depth indicate that GRP78 has relation with the growth of cancer cells. The correlations between GRP78 overexpression and metastasis to lymph nodes and extracapsular spread in lymph nodes indicate that GRP78 has relation with the metastasis of cancer cells. Thus, it is clinically proved that GRP78 participates in the growth and metastasis of cancer cells. Therefore, the present invention uses the molecule GRP78 in clinically prognosing the tumor grade of head and neck cancer.

TABLE 2

| | | GRP78 overexpression (%) | | | | | |
|---|---|---|---|---|---|---|---|
| Parameters | | N | No | | Yes | | P value |
| T stage | T1-t2 | 30 | 14 | (47) | 16 | (53) | 0.035 |
| | T3-t4 | 29 | 6 | (21) | 23 | (79) | |
| N stage | N = 0 | 44 | 19 | (43) | 25 | (57) | 0.010 |
| | N > 0 | 15 | 1 | (7) | 14 | (93) | |
| Overall stage | I-II | 27 | 13 | (48) | 14 | (52) | 0.034 |
| | III-IV | 32 | 7 | (22) | 25 | (78) | |
| Differentiation | Benignant | 24 | 8 | (33) | 16 | (67) | 0.939 |
| | *m-p | 35 | 12 | (34) | 23 | (66) | |
| Tumor depth | <10 mm | 30 | 14 | (47) | 16 | (53) | 0.035 |
| | >10 mm | 29 | 6 | (21) | 23 | (79) | |
| Extracapsular | No | 47 | 19 | (40) | 28 | (60) | 0.036 |
| spread in lymph | Yes | 12 | 1 | (8) | 11 | (92) | |
| nodes | | | | | | | |
| Sum | | 59 | 20 | | 39 | (66) | |

*m-p: moderate to poor

Those described above are only to exemplify the present invention but not to limit the scope of the present invention. Any modification or variation according to the spirit of the present invention is to be also included within the scope of the present invention, which is based on the claims stated below.

What is claimed is:

1. A method for prognosing a tumor grade of head and neck cancer, characterized in using a 78-kDA glucose regulated protein (GRP78) as a labeled molecule of a head and neck cancer, wherein relative expression of said 78-kDA glucose regulated protein in said head and neck cancer tissue and a normal tissue is used to prognose a tumor grade of said head and neck cancer.

2. The method for prognosing a tumor grade of head and neck cancer according to claim 1, wherein said relative expression of said 78-kDA glucose regulated protein in said head and neck cancer tissue and said normal tissue has a positive correlation with said tumor grade of said head and neck cancer.

3. The method for prognosing a tumor grade of head and neck cancer according to claim 1, wherein said tumor grade of said head and neck cancer includes factors of tumor size, tumor depth and tumor metastasis to nymph nodes.

4. The method for prognosing a tumor grade of head and neck cancer according to claim 1, wherein a protein analysis is used to detect relative expression of said 78-kDA glucose regulated protein in said head and neck cancer tissue and said normal tissue.

5. The method for prognosing a tumor grade of head and neck cancer according to claim 4, wherein a western blot analysis method is used to perform said protein analysis of said 78-kDA glucose regulated protein in said head and neck cancer tissue and said normal tissue.
